(19) United States
(12) Patent Application Publication
(10) Pub. No.: US 2012/0070889 A1
(43) Pub. Date: Mar. 22, 2012

(54) HYBRID BIOREACTOR FOR REDUCTION OF CAPITAL COSTS

(75) Inventor: David J. Bayless, Athens, OH (US)

(73) Assignee: OHIO UNIVERSITY, Athens, OH (US)

(21) Appl. No.: 13/322,682

(22) PCT Filed: May 27, 2010
(86) PCT No.: PCT/US10/36297

§ 371 Date: Nov. 28, 2011

Related U.S. Application Data
(60) Provisional application No. 61/181,682, filed on May 28, 2009.

(51) Int. Cl. C12M 1/00

(52) U.S. Cl. 435/292.1

(57) ABSTRACT

A bioreactor with first and second containers has translucent sidewalls housing a plurality of planar membranes over which a slurry of water and photosynthetic microorganisms flows. A light collector is mounted adjacent a gap between the first and second containers for receiving incident light and conveying the light through optical waveguides, such as fibers, to distributors. The distributors are mounted in the first and second containers near a lower end of the substrates so that photons incident on the optical waveguides are conveyed through the optical waveguides to the light distributors and then to the lower ends of the substrates where light passing through the translucent sidewalls is less intense.

### BACKGROUND OF THE INVENTION

This invention relates to an apparatus and a method for growing photosynthetic microorganisms, such as algae. One implementation is to use the apparatus to sequester carbon dioxide in exhaust gas, thereby providing a gas cleaning systems for fossil burning plants.

It is well known that fossil fuels, such as petroleum-derived fuels and coal, are limited in supply. Additionally, the combustion of such fuels contributes substantial carbon to the atmosphere. The release of carbon long stored in such fuels is the subject of global concern relating to climate change and other environmental problems. Nevertheless, fossil fuels are the largest fuel source for automobiles and energy production facilities.

The U.S. produces an estimated 1.7 billion tons of CO₂ annually from the combustion of fossil fuels. Even if an expensive option for CO₂ removal is discovered, CO₂ “disposal” is problematic. U.S. industries consume only 40 million tons of CO₂, produced at a much lower price than possible by removing CO₂ from flue gas, which is the exhaust gas from a fossil fuel burning power plant. Therefore, increased consumption of CO₂ appears limited, and options for expanded use appear limited and costly.

Existing power plants have capital values in the hundreds of billions of dollars, and sequestration of CO₂ is necessary in order to keep the plants operating without releasing more CO₂ into the atmosphere. A feasible option involves biological carbon sequestration in outdoor ponds near the power plants. However, there are inherent inefficiencies related to this solution for CO₂ sequestration, primarily due to the amount of cyanobacteria that can be grown in a given volume. For example, if 2,000,000 m² of photosynthetic surface area is required for 25% reduction of CO₂ emissions from a power plant, that is equivalent to almost 500 acres of surface. Very few existing plants have 500 acres available to them and fewer could afford to convert 500 acres to a shallow lake or raceway cultivator. Also, there are serious questions about how to distribute the flue gas (or separated CO₂) into the lake for maximum growth, not to mention what to do with the gas once it bubbles to the surface. The flue gas would have to be collected again and redirected up a stack to meet other emission requirements. Further, maintaining such a large “lake” during a Midwestern winter would introduce more practical problems.

Clearly, other approaches for CO₂ control are needed. Research to develop a robust portfolio of carbon management options, including safe and effective photosynthetic carbon recycling, will enable continued use of coal in electrical power generation. Despite the large body of research in this area, virtually no work has been done to create a practical system for greenhouse gas control, one that could be used with both new and existing fossil units.

One area of increased interest for carbon sequestration is in biofuels, which are derived from recently living organisms or their metabolic byproducts. Biofuels contain different hydrogen and carbon-containing molecules than fossil fuels, and sufficient enthalpy to compete with fossil fuels for vehicle fuel and energy production. Most biofuels are considered neutral in their release of carbon into the atmosphere. This is due to the fact that, even though the living organisms remove carbon from the air, that carbon is subsequently released during the chemical reaction that produces work from the stored solar energy.

Biofuels are a renewable energy source, unlike other natural resources such as petroleum, coal, and nuclear fuels. For example, some biofuels can be grown in a conventional setting, such as a farm field, while others must be grown in unique, controlled settings. A bioreactor is a vessel in which a chemical process is carried out that involves organisms or biochemically active substances derived from such organisms. Known bioreactors take the exhaust gases of, for example, fossil fuel burning power plants, and use the CO₂ therein to “fuel” growth of microalgae and other photosynthetic microorganisms. Such bioreactors prevent carbon from the exhaust gas stream from being released into the air, and produce biofuels that provides additional energy. Open-pond bioreactor systems have existed for some time, but are unsuitable in many ways, especially for large sources of CO₂.

Microalgae have much faster growth-rates than terrestrial crops. Depending on the bioreactor and the strain, the per unit area yield of oil from algae is estimated to be many times greater than the next best crop, which is palm oil. Algal-oil processes into biodiesel as easily as oil derived from land-based crops. The difficulties in efficient biodiesel production from algae lie in finding a cost-effective bioreactor that is best suited to a strain of algae that contains sufficient lipids.

Microalgae are organisms capable of photosynthesis that are less than 2 mm in diameter. These include the diatoms and cyanobacteria. This preference towards microalgae is due largely to its less complex structure, fast growth rate, and high oil content in some species.

Despite the scientific advantages of biofuels and the availability of bioreactors that are capable of producing such fuels and sequestering carbon, economic disadvantages have restricted the extent to which bioreactors have been implemented. For example, one disadvantage of conventional bioreactors is the fact that they become economically feasible only when natural light is used. The ability to expose microorganisms to sufficient natural light is a function of the exposed surface area of conventional bioreactors. Space is not always available where large supplies of CO₂ are being produced. Biofuels produced from such bioreactors can only compete with petroleum-based fuels if their production is high enough that economies of scale exist. This is difficult with conventional bioreactors.

The production of microalgae as a feedstock for mitigation of carbon dioxide emission and production of biofuels requires environmentally controlled bioreactors that are capable of maximum productivity for the lowest possible capital cost. Ohio University has created an interesting and viable bioreactor system based upon light collection and distribution from external sources. This system optimizes the use of collected photons, but significantly increases the capital cost.

Therefore, the need exists for a bioreactor that makes carbon sequestration and biofuel production economically feasible.

### BRIEF SUMMARY OF THE INVENTION

An object of the invention is a high degree of algal productivity and environmental control using a hybrid arrangement of light collection and distribution, while minimizing the capital cost for the overall system. The bioreactor according to the invention is illuminated with a combination of collected and “natural” (direct) light sources, making it a hybrid system. The invention is thus not as efficient as one that is illuminated from collected and specifically distributed light. However, it allows for vast increases in productivity over a system that depends totally on incident sunlight, such as a greenhouse, raceway cultivator or pond, and does so at substantially less cost compared to one that is illuminated only from collected and specifically distributed light. The arrangement of the light collection hardware to the “greenhouse” containment reactor is important to the operation of bioreactors made according to the invention, so as to maximize the photosynthetically active radiation available to the microalgae or cyanobacteria and thereby produce significant photosynthetic activity.

The invention eases the transition from the least effective, lowest capital cost option for growing cyanobacteria and microalgae (the open pond) to the more efficient system (the Ohio University bioreactor described in U.S. Pat. No. 6,667,171, which is incorporated herein by reference). The hybrid bioreactor of the invention uses incident light directly from the sun and also uses collected and distributed light to make up for the significant lack of penetration and distribution that would result from using only incident sunlight. This provides an excellent balance of costs and efficiency, as compared to the existing systems.

This invention was made with Government support under contract DE-FG36-08GO88083 awarded by the U.S. Department of Energy. The Government has certain rights in the invention.

### BRIEF DESCRIPTION OF THE SEVERAL VIEWS OF THE DRAWINGS

FIG. 1 is a schematic perspective view illustrating a bioreactor and collector combination according to the present invention with all solid regions shaded to make them visible.

FIG. 2 is a schematic perspective view illustrating the bioreactor and collector combination shown in FIG. 1 with all solid regions left transparent to more easily view the interior of the apparatus.

FIG. 3 is a schematic perspective view illustrating the bioreactor and collector combination shown in FIG. 1 in “open” photobioreactor depending only on sun for light in early morning.

FIG. 4 is a schematic perspective view illustrating the bioreactor and collector combination shown in FIG. 1 in “open” photobioreactor depending only on sun for light in mid-morning.

FIG. 5 is a schematic perspective view illustrating the bioreactor and collector combination shown in FIG. 1 in “open” photobioreactor depending only on sun near solar noon.

FIG. 6 is a schematic perspective view illustrating the bioreactor and collector combination shown in FIG. 1 in “open” photobioreactor depending only on sun for light in mid-afternoon.

FIG. 7 is a schematic perspective view illustrating the bioreactor and collector combination shown in FIG. 1 in “open” photobioreactor depending only on sun for light near evening.

FIG. 8 is a schematic perspective view illustrating the bioreactor and collector combination shown in FIG. 1 with the addition of fiber optic or other light distributors at the bottom to increase photon distribution.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

This application claims the benefit of U.S. Provisional Application No. 61/181,682 filed May 28, 2010. This priority application is hereby incorporated by reference.

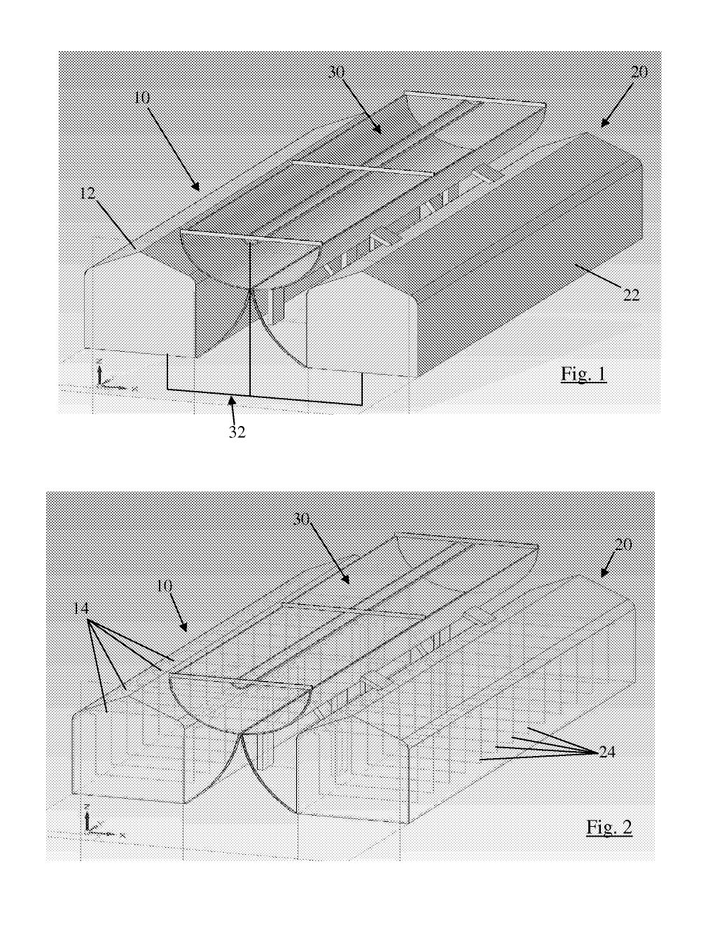

The preferred embodiment of the present invention is shown in FIGS. 1 and 2. In FIG. 1, two open “greenhouse” type bioreactors 10 and 20 are shown with sidewalls 12 and 22, respectively. The terms “sidewalls” is used broadly to include any exterior containment structure, such as the wall, floor and ceiling of the bioreactors 10 and 20. As shown in FIG. 2, in which the sidewalls 12 and 22 are transparent, multiple suspended growth membranes 14 and 24 are mounted in the bioreactors 10 and 20, respectively. The growth membranes are preferably of the type described in U.S. Pat. No. 6,667,171, which is incorporated herein by reference. Thus, a mechanism is provided to flow water, nutrients and microorganisms in and across the membranes 14 and 24 within the bioreactors to promote the exposure of the organisms to light, thereby enabling and enhancing growth of the organisms. The membranes and water flow mechanism are conventional, and are similar or equivalent to those disclosed in U.S. Pat. No. 6,667,171.

In the preferred embodiment, all or most of the sidewalls 12 and 22 of the bioreactors 10 and 20 are translucent (or transparent) to permit a significant amount of light, preferably sunlight, incident upon the sidewalls 12 and 22 to penetrate through the sidewalls, thereby striking the interior surfaces within the bioreactors 10 and 20, including the membranes 14 and 24. The sidewalls 12 and 22 thus allow “natural” daylight to provide some of the photons required for photosynthetic growth on the membranes. By making at least one of the sidewalls (such as the ceiling and vertical walls) of the bioreactor container translucent, light incident thereon is allowed to pass through to the substrates without substantial loss of intensity.

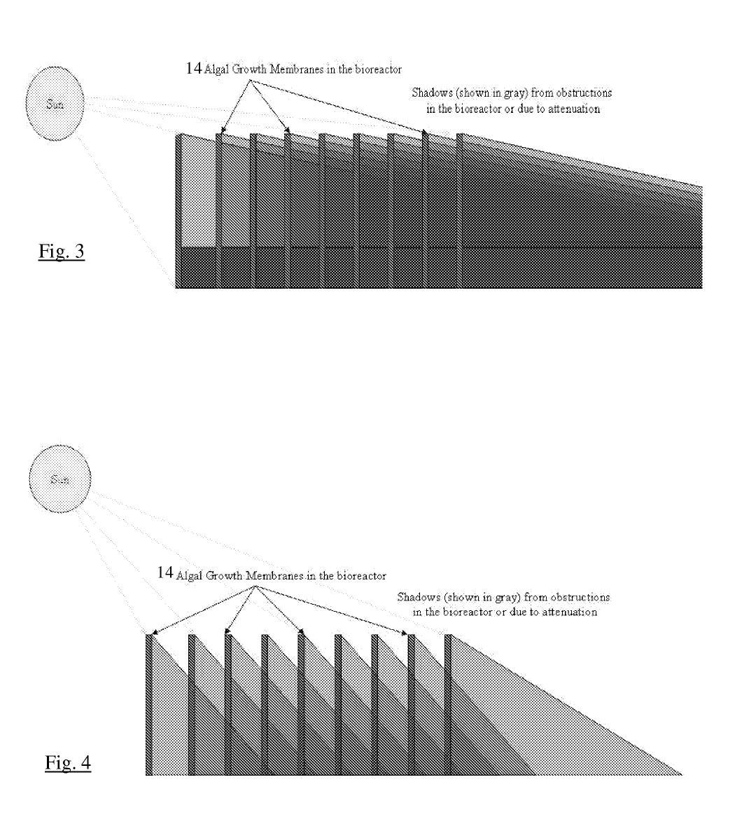

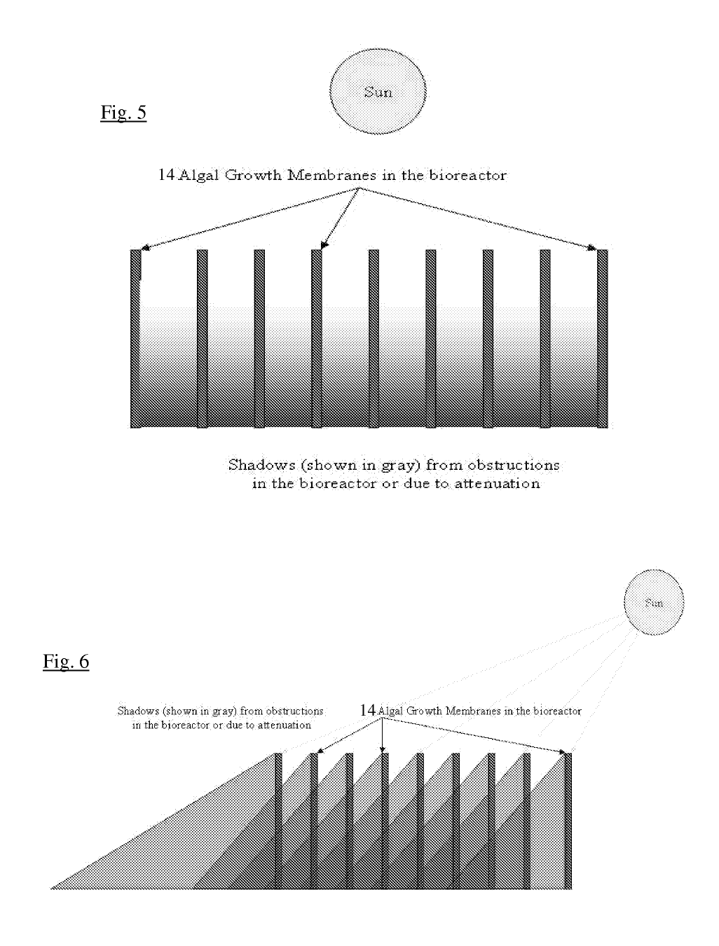

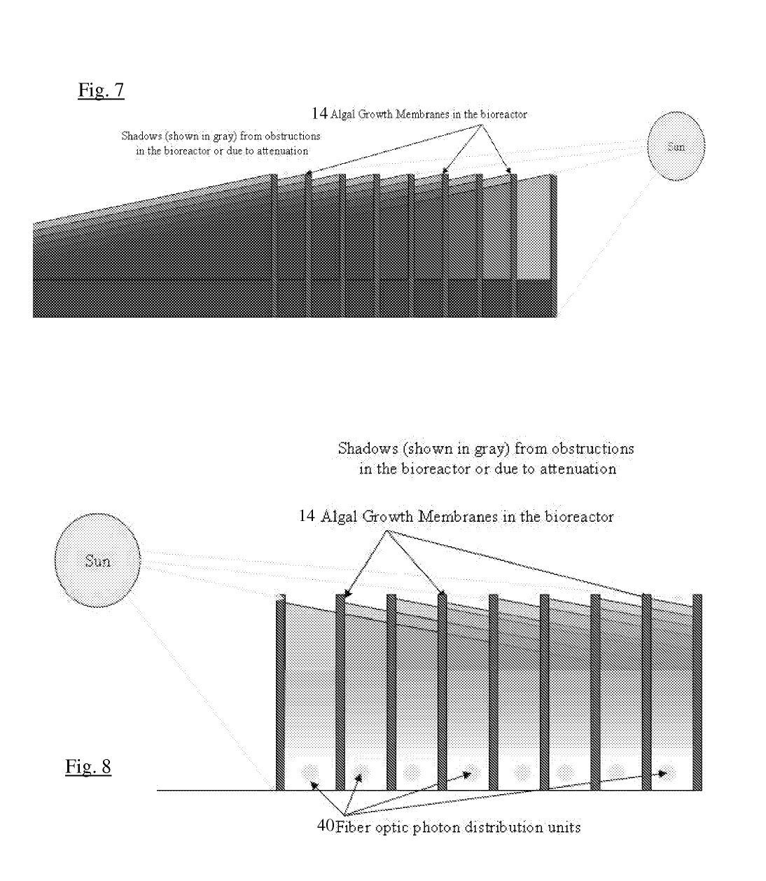

Using the translucent sidewalls 12 and 22 alone, a bioreactor would have light incident on the membranes 14 and 24 as shown in FIGS. 3-7, and would do so at a low cost. This would provide benefits due to the light striking the membranes 14 and 24 directly, but there would still be a problem due to the large area at the bottoms of each membrane when the sun is at virtually any angle other than at or near solar noon (FIG. 5). As shown in FIGS. 3-4 and 6-7, significant shading of at least portions of the membranes occurs in the morning and evening.

As shown in FIGS. 1 and 2, a solar tracking trough 30 is located adjacent a gap formed between the spaced bioreactors. The trough 30 is elevated above the bioreactors to collect light from the sun in a conventional manner and channel it to increase photon incidence where desired. The position of the trough 30 between the bioreactors, combined with its elevated location minimizes interference with sunlight incident at the high solar angles (at and near solar noon), and provides at least some obstruction of sunlight incident at the extreme morning and evening angles, particularly to those upper portions of the membranes 14 and 24 that are the most likely to be overexposed. Thus, the trough 30 provides lighting benefits at all times, and, due to its position, it provides the least interference when the external light can penetrate the deepest into the bioreactor. In FIG. 5 the position of the sun relative to the membranes 14 is illustrated at solar noon when the degree of penetration through the sidewalls (the sidewalls are not shown in FIGS. 3-8) is greatest. The trough 30, though not illustrated in FIGS. 3-8, provides little obstruction to sunlight shining on the membranes 14 when the sun is at or near solar noon.

Optical waveguides, such as conventional fiber optic cables 32 (see FIG. 1), connect to the collecting mirror apparatus of the trough 30 and extend to light distributors 40 (not visible in FIG. 1), such as notched fibers or etched glass poles, at the bottom of the bioreactors 10 and 20 as shown in FIG. 8. The distributors 40 provide light from the “bottom up” in the bioreactor, negating the shade to increase photon distribution onto the membranes 14 and 24. The distributors 40 are preferably mounted at or near the bottom of the vertically mounted bioreactor between the membranes as shown in FIG. 8 or otherwise strategically placed in order to have the desired effect of distributing light over the lower portion of the membranes 14 and 24 or other substrate used. It is preferred that each of the distributors 40 directs light onto the lower 70 percent of the height of the membranes 14 and 24. More preferably, the distributors direct light on to the lower 50 percent of the membranes, and most preferably the lower 30 percent of the membranes.

It should be noted that the invention does not apply only to vertical membrane photobioreactors. The invention has application in any bioreactor using vertical containment, including plastic bag bioreactors and tube bioreactors known to persons having ordinary skill All bioreactors that use vertical substrates or containment have problems with self-shading, attenuation when the algal colony gets too dense or thick, and ineffective light distribution. Vertically-mounted bioreactors have problems with either too much light incident on the substrates or containment (creating photo-inhibition), or too little light incident on the substrates or containment (resulting in limited growth of the microorganisms).

The combination of the invention provides the benefits of low cost and high light incidence on the substrates. The combination of a transparent exterior photobioreactor and an apparatus that collects and distributes light to the lower regions of the substrates provides these benefits. Significant growth of the microorganisms can be achieved at much less cost using a suspended growth membrane photobioreactor with “open” and collected and distributed sunlight, than with embodiments using an opaque exterior and delivering all photosynthetic active radiation from collected and distributed photons. The distributed lighting in the areas of the bioreactor receiving low light incidence in morning and evening (seen in FIGS. 3-4 and 6-7) causes microalgae to grow at desired productivities even with low light penetration at times other than solar noon.

The use of the conventional collecting system having a tracking system with minors and distribution using fiber optics to carry photosynthetically active radiation into the “dark” regions of the bioreactor more than doubles the incident photons in the bioreactor on a daily basis, when compared to transparent bioreactors alone. Also important is the fact that the particular trough 30 apparatus between pairs of bioreactors optimizes the distribution of photons. Areas of the bioreactors that experience too high a level of photons due to natural sunlight are partially shaded by the trough 30 in the morning and evening. Furthermore, areas of the bioreactors that would otherwise be too dark with a transparent exterior alone receive photons at a favorable flux to optimize photosynthetic efficiency and productivity in the overall bioreactor.

The shape and size of the bioreactors 10 and 20 is not critical, and it will become apparent to a person having ordinary skill that other shapes and sizes can be substituted for those shown and described. Furthermore, the use of the collecting trough 30 is advantageous, but those of ordinary skill will know of other collecting and distributing structures that could be substituted for that shown in the preferred embodiment.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A bioreactor apparatus comprising:
(a) a first container having sidewalls, a floor and a ceiling defining a chamber housing at least one substrate over which a slurry of at least water and photosynthetic microorganisms flows, wherein at least one of the sidewalls and ceiling is substantially translucent, thereby permitting light incident thereon to pass through to said at least one substrate without substantial loss of intensity;
(b) a light collector for receiving light incident thereon and focusing the light onto a first end of at least one optical waveguide; and
(c) a first light distributor mounted in the first container near a lower end of said at least one substrate and connected to a second end of said at least one optical waveguide, wherein at least some photons incident on the first end of said at least one optical waveguide are conveyed through the optical waveguide and the light distributor to said at least one substrate.

2. The apparatus in accordance with claim 1, wherein said at least one substrate further comprises a plurality of substantially planar membrane substrates.

3. The apparatus in accordance with claim 2, further comprising:
(a) a second container, spaced from the first container to form a gap, the second container having sidewalls, a floor and a ceiling defining a second chamber housing a plurality of substantially planar membrane substrates over which a slurry of at least water and photosynthetic microorganisms flows, wherein the sidewalls and ceiling are substantially translucent, thereby permitting light incident thereon to pass through to at least some of the substrates without substantial loss of intensity; and
(b) a second light distributor mounted in the second container near a lower end of the substrates therein and connected to a second end of a second optical waveguide, wherein at least some photons incident on the first end of the second optical waveguide are conveyed through the optical waveguide and the second light distributor to the substrates.

4. A bioreactor apparatus comprising:
(a) a first container having sidewalls, a floor and a ceiling defining a chamber housing a plurality of substantially planar membrane substrates over which a slurry of at least water and photosynthetic microorganisms flows, wherein the sidewalls and ceiling are substantially translucent, thereby permitting light incident thereon to pass through to at least some of the substrates without substantial loss of intensity;
(b) a second container, spaced from the first container to form a gap, the second container having sidewalls, a floor and a ceiling defining a second chamber housing a plurality of substantially planar membrane substrates over which a slurry of at least water and photosynthetic microorganisms flows, wherein the sidewalls and ceiling are substantially translucent, thereby permitting light incident thereon to pass through to at least some of the substrates without substantial loss of intensity;
(c) a light collector mounted adjacent the gap between the first and second containers for receiving light incident thereon and focusing the light onto a first end of at least first and second optical waveguides;
(d) a first light distributor mounted in the first container near a lower end of the substrates therein and connected to a second end of the first optical waveguide, wherein at least some photons incident on the first end of the first optical waveguide are conveyed through the optical waveguide and the first light distributor to the substrates; and
(e) a second light distributor mounted in the second container near a lower end of the substrates therein and connected to a second end of the second optical waveguide, wherein at least some photons incident on the first end of the second optical waveguide are conveyed through the optical waveguide and the second light distributor to the substrates.

5. The apparatus in accordance with claim 4, wherein the substrates in the first container are substantially parallel and equally spaced, the first light distributor comprises a plurality of light poles, each of the light poles is mounted in a gap between pairs of the substrates in the first container, the substrates in the second container are substantially parallel and equally spaced, the second light distributor comprises a plurality of light poles, and each of those light poles is mounted in a gap between pairs of the substrates in the second container.
